(19) United States
(12) Patent Application Publication
(10) Pub. No.: US 2004/0166512 A1
(43) Pub. Date: Aug. 26, 2004

(54) Method for cloning PCR products without restriction or ligation enzymes

(76) Inventors: Michael L. Kahn, Pullman, WA (US); Brent L. House, Pullman, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/705,721

(22) Filed: Nov. 10, 2003

Related U.S. Application Data
(60) Provisional application No. 60/425,955, filed on Nov. 12, 2002.

(51) Int. Cl. C12Q 1/68; C12P 19/34; C12N 15/74

(52) U.S. Cl. 435/6; 435/91.2; 435/488

(57) ABSTRACT

In one aspect the invention provides methods for cloning polymerase chain reaction (PCR) products without the need for restriction enzymes, ligation enzymes, or DNA purification steps. According to these methods, a PCR product is transferred into a vector in vivo using a site-specific recombination system. In some embodiments, the methods include the steps of (1) providing a PCR product flanked by a first site-specific recombination site and a second site-specific recombination site; and (2) transferring the PCR product into a cell comprising a target sequence flanked by a first recombination site partner and a second recombination site partner, and at least one recombination protein that mediates recombination between the first site-specific recombination site and the first recombination site partner, and between the second site-specific recombination site and the second recombination site partner.

### CROSS-REFERENCE TO RELATED APPLICATION

This application claims the benefit of U.S. Provisional Application No. 60/425,955, filed Nov. 12, 2002.

### GOVERNMENT RIGHTS

This invention was made with government support awarded by the National Science Foundation (Grant No. MCB-0133258). The government has certain rights in the invention.

### FIELD OF THE INVENTION

The present invention relates to methods for cloning polymerase chain reaction products using site-specific recombination.

### BACKGROUND OF THE INVENTION

The use of the polymerase chain reaction (PCR) in nucleic acid research has provided a convenient way to amplify and construct recombinant DNA. PCR is an in vitro method of nucleic acid synthesis by which a particular segment of DNA can be specifically replicated. The method involves two oligonucleotide primers that flank the DNA fragment to be amplified and repeated cycles of heat denaturation of the DNA, annealing of the primers to their complementary sequences, and extension of the annealed primers with DNA polymerase. These primers hybridize to opposite strands of the target sequence, the primers being oriented so that DNA synthesis by the polymerase proceeds across the region between the primers. Since the extension products themselves are also complementary to and capable of binding primers, successive cycles of amplification essentially double the amount of DNA synthesized in the previous cycle. The result is an exponential accumulation of the target fragment, approximately 2ⁿ, where n is the number of cycles of amplification performed.

For most applications, PCR products need to be cloned into a vector. Cloning of PCR products into a vector generally involves the incorporation of restriction sites at the ends of PCR products or blunt-ended ligation of PCR products into the vector.

Several technical problems exist with the use of such techniques for cloning PCR products. Restriction sites may exist within the DNA to be cloned and provide targets for cleaving the DNA to be inserted. Restriction sites at the ends of PCR products are often inefficiently cleaved by restriction enzymes because they are too close to the end of the DNA fragment. The PCR products lack part of the binding region for the restriction enzyme to contact the DNA template. The introduction of extraneous nucleotides at the 3′ end of the amplified DNA fragments by Taq DNA polymerase leads to relatively low overall efficiency of the blunt-end ligation reaction. The incorporation of extraneous nucleotides would alter a reading frame and constructs a copy that includes the undesired nucleotide. The efficiency of blunt ended ligation is poor for the following reasons: i) the Kₘ for the activity of T4 ligase on blunt-ended DNA is nearly 100 times higher than its Kₘ on DNA with cohesive ends, thus, ligation of blunt-ended DNA requires a high concentration of enzyme and a high concentration of DNA ends (greater than 1 microgram), therefore very large amounts of the fragment to be cloned are needed, ii) during blunt-end ligation, a fraction of the plasmid vector will recircularize and contribute to non-recombinant backgrounds, and iii) because of the high concentration of the fragments to be cloned, many recombinant plasmids will contain more than one insert of foreign DNA. Furthermore, blunt ended cloning is directionless. In many applications, the orientation of the DNA fragment in the PCR construct is crucial for gene expression.

Thus, cloning of PCR products is often not straightforward. Moreover, it generally requires several steps, including DNA fragment purification, an overnight ligase-dependent ligation and colony selection to determine the correct orientation of the insert. These steps are usually labor intensive, time consuming and/or of low efficiency. It will usually take at least one day to prepare DNA fragments and another day for the ligase reaction.

There is a need for more efficient methods for cloning PCR products that do not require the use of restriction enzymes, ligation enzymes, or DNA purification steps.

### SUMMARY OF THE INVENTION

In one aspect the invention provides methods for cloning polymerase chain reaction (PCR) products into a target sequence that do not require the use of restriction or ligation enzymes in vitro, or DNA purification steps. According to the invention, the PCR products may be cloned directly from the PCR reaction. According to these methods, a PCR product is cloned into a target sequence in vivo using a site-specific recombination system. In some embodiments, the methods include the steps of (1) providing a PCR product flanked by a first site-specific recombination site and a second site-specific recombination site; and (2) transferring the PCR product into a cell comprising a target sequence flanked by a first recombination site partner and a second recombination site partner, and at least one recombination protein that mediates recombination between the first site-specific recombination site and the first recombination site partner, and between the second site-specific recombination site and the second recombination site partner.

The PCR products are typically generated using a set of primers to provide the first and second recombination sites to the PCR product. In some embodiments, the PCR products are linear. The target sequence may be a genomic sequence or a plasmid sequence. The target sequence may be transferred into the cell simultaneously with the PCR product.

In some embodiments, the site-specific recombination system is the integrase/attachment system from bacteriophage lambda. In some embodiments, the cell is an E. coli bacterial cell. The PCR products may be transferred into a cell that further comprises at least one other site-specific recombination protein, such as Integration Host Factor.

In a further aspect, the invention provides site-specific recombination kits including vectors, primers, and cells for cloning PCR products in vivo.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a graph comparing the number of clones (colony forming units, CFU) obtained after transformation as a function of cell density (OD600) at the time of harvesting cells, as described in EXAMPLE 1. Transformation frequency of E. coli with supercoiled control DNA is shown as “Δ - -”, and transformation frequency with vector and PCR product containing the attB sequences leading in vivo recombination is shown as “-♦-”. The right y-axis represents the number of clones obtained with the control DNA, the left y-axis represents the number of clones obtained using the PCR product.

### DETAILED DESCRIPTION OF THE PREFERRED EMBODIMENT

Unless specifically defined herein, all terms used herein have the same meaning as they would to one of ordinary skill in the art.

In one aspect, the invention provides methods for cloning polymerase chain reaction (PCR) products by site-specific recombination in vivo. Site-specific recombinases are proteins that are present in many organisms (e.g., viruses and bacteria) and have been characterized to have both endonuclease and ligase properties. These recombinases (along with associated proteins in some cases) recognize specific sequences of bases in DNA and exchange the DNA segments flanking those segments. The recombinases and associated proteins are collectively referred to as “recombination proteins” or “recombination proteins and accessory proteins” (see, e.g., Landy, A. (1993) Curr. Op. Biotechnol. 3:699-707).

Numerous site-specific recombination systems from various organisms have been described (see, e.g., Hoess et al. (1986) Nucleic Acids Research 14(6):2287; Abremski et al. (1986) J. Biol. Chem. 261(1):391; Campbell (1992) J. Bacteriol. 174(23):7495; Qian et al. (1992) J. Biol. Chem. 267(11):7794; Araki et al. (1992) J. Mol. Biol. 225(1):25; Maeser & Kahnmann (1991) Mol. Gen. Genet. 230:170-176; Esposito et al. (1997) Nucl. Acids Res. 25(18):3605). Many of these belong to the integrase family of recombinases (Argos et al. (1986) EMBO J. 5:433-440). Perhaps the best studied of these are the Integrase/att system from bacteriophage lambda (Landy (1993) Curr. Op. Genet. Devel. 3:699-707), the Cre/loxP system from bacteriophage P1 (Hoess & Abremski (1990) In Nucleic Acids Mol. Biol. 4:90-109; Eckstein & Lilley, eds, Berlin-Heidelberg, Springer-Verlag), and the Flp/FRT system from the Saccharomyces cerevisiae 2μ circle plasmid (Broach et al. (1982) Cell 29:227-234).

The major advantage of using integrases is that, unlike cut and paste technology with restriction enzymes and DNA ligase, integrases catalyze a concerted recombination. The very long sequence-specificity of the integrase reaction allows them to catalyze precise manipulations with large DNA molecules that would be difficult with restriction enzyme techniques.

The prototypical site-specific recombination reaction is used to integrate the lysogenic bacteriophage, lambda, into the E. coli chromosome. Two DNA sequences are involved, attP, the 243 bp phage attachment sequence, and attB, the 25 bp bacterial site of attachment (Mizuuchi & Mizuuchi (1980) Proc. Natl. Acad. Sci. U.S.A. 77:3220-4; Mizuuchi & Mizuuchi (1985) Nucleic Acids Res. 13:1193-208). Recombination catalyzed by the integrase protein, Int, occurs within a 15 bp “core” region of identity between attP and attB. Recognition of these sequences by integrase primarily involves the “arm” sequences of attP, which extend about 150 bases to the left and 100 bases to the right of the core (Mizuuchi & Mizuuchi (1980) Proc. Natl. Acad. Sci. U.S.A. 77:3220-4). If the core sequence in attP is replaced with another sequence, and there is an identical sequence on another DNA molecule that can pair with the new core, recombination can also take place. Integration causes the phage chromosome to be covalently attached to the bacterial chromosome, flanked by hybrid sequences called attL and attR. This is the BP reaction, because it involves attB and attP as substrates. The host integration host factor (IHF) is also required (Miller et al. (1980) Cell 20:721-9). The BP reaction requires the attP DNA to be supercoiled but attB DNA can be linear, like a PCR product.

Reversal of the reaction, used to excise lambda from the chromosome, additionally involves the phage protein Xis and restores the original attB and attP sequences. This LR reaction does not require supercoiled DNA and, in fact, works much better when the DNA is relaxed or linear. The BP and LR reactions are well characterized, function at room temperature, involve relatively stable proteins, and are robust.

Another site-specific recombination system is the yeast Flp recombinase, which is used in a recombination coupled to replication of the yeast 2μ plasmid (Sadowski (1995) Prog. Nucleic Acid Res. Mol. Biol. 51:53-91). Unlike integrase, the yeast Flp recombinase functions primarily in one direction, catalyzing recombination between two DNA sequences (FRT) that are antiparallel in the plasmid and thereby inverting the sequences between them. In the most common biotechnological applications of Flp/FRT, the FRT sequences are set up in parallel orientation and therefore the reaction leads to excision of the DNA between them (Hoang et al. (1998) Gene 212:77-86; Theodosiou et al. (1998) Methods 14:355-65). Accordingly, some embodiments of the methods and the vectors of the invention use the lambda or the Flp site-specific recombination systems. The use of other site-specific recombination systems is also within the spirit and scope of the invention.

In one aspect, the invention provides methods for cloning PCR products without the need for in vitro provision of restriction enzymes, ligation enzymes, or DNA purification steps. According to these methods, a PCR product is cloned in vivo using a site-specific recombination system. In some embodiments, the methods include the steps of (1) providing a PCR product flanked by a first site-specific recombination site and a second site-specific recombination site; and (2) transferring the PCR product into a cell comprising a target sequence flanked by a first recombination site partner and a second recombination site partner, and at least one recombination protein that mediates recombination between the first site-specific recombination site and the first recombination site partner, and between the second site-specific recombination site and the second recombination site partner.

The PCR product used in the methods of the invention typically is linear. Linear DNA has been previously used to transform bacterial cells, such as Bacillus subtilis (Contente & Dubnau (1979) Plasmid 2(4):555-71), Lactobacillus plantarum (Thompson et al. (1997) Lett. Appl. Microbiol. 35(6):419-25), and E. coli (Hoekstra et al. (1980) J. Bacteriol. 143(2):1031-2). However, these methods have involved either transforming DNA possessing an origin of replication and selecting for recircularization of the plasmid DNA (Conley & Saunders (1984) Mol. Gen. Genet. 194(1-1):211-8; Conley (1986) Nucl. Acids Res. 14(22):8919-32), or transforming DNA containing regions of homology with the host organism's genome, and selecting for integration of the DNA into the chromosome or a plasmid containing homologous DNA (Dabert & Smith (1997) Genetics 145(4):877-89; Yu et al. (2000) Proc. Natl. Acad. Sci. U.S.A. 97(11):5978-83; Lee et al. (2001) Genomics 73(1):56-65). In contrast, the methods of the invention use site-specific recombination.

In the methods of the invention, at least three elements are brought together in a cell to clone a PCR product into a target sequence: (1) a PCR product flanked by a first and second recombination sites; (2) a target sequence flanked by a first and second recombination site partners; and (3) one or more recombination proteins to mediate recombination between the recombination sites and the recombination site partners. Moreover, the genetic background of the recipient cell should be conducive to recombination. For example, the use of mutant cells that are missing a protein that might interfere with recombination, such as recD, may be desirable.

The PCR product used in accordance with the invention typically includes two site-specific recombination sites which allow the PCR product to be transferred into one or more target sequences. The PCR product is typically generated using a set of primers to provide the first and second recombination sites to the PCR product. The PCR product may be any nucleic acid molecule derived from any source and may include non-naturally occurring nucleic acids. Additionally, the PCR product may comprise a particular sequence of interest (e.g., a gene) or it may comprise a population of molecules (e.g., molecules generated from genomic or cDNA libraries).

In some embodiments, libraries (e.g., populations of genomic DNA or cDNA, or populations of nucleic acid molecules, produced by de novo synthesis such as random sequences or degenerate oligonucleotides) are utilized in accordance with the present invention. By the recombination methods of the invention, the library may be easily inserted into different target sequences. These target sequences may be genomic sequences or they may be on plasmid vectors (or combinations of vectors). Such vectors may be introduced into different host systems (prokaryotic and eukaryotic) to evaluate and analyze the library or a particular sequences or clones derived from the library. Alternatively, vectors containing the cloned PCR products may be used in vitro systems such as in vitro expression systems for production of RNA and/or protein.

The target sequence for site-specific recombination of the PCR product according to the methods of the invention may be a genomic sequence, or it may be a plasmid sequence. The methods of the invention can be used with any genomic target sequences, prokaryotic or eukaryotic. Similarly, the methods may be used with target sequences on plasmid vectors that may function in a variety of systems or host cells, for example, prokaryotic vectors, eukaryotic vectors or vectors which may shuttle between various prokaryotic and/or eukaryotic systems (e.g., shuttle vectors). Prokaryotic plasmid vectors for use in the invention include but are not limited to vectors which may propagate and/or replicate in gram negative and/or gram positive bacteria, including bacteria of the genus Escherichia, Salmonella, Proteus, Clostridium, Klebsiella, Bacillus, Streptomyces, and Pseudomonas, Sinorhizobium, and E. coli. Eukaryotic vectors for use in the invention include vectors which propagate and/or replicate and yeast cells, plant cells, mammalian cells (e.g., human cells), fungal cells, insect cells, fish cells and the like. Particular plasmid vectors of interest include but are not limited to cloning vectors, sequencing vectors, expression vectors, fusion vectors, two-hybrid vectors, gene therapy vectors, and reverse two-hybrid vectors. Such vectors may be used in prokaryotic and/or eukaryotic systems depending on the particular vector.

Typically, the target sequence comprises a marker gene that permits identification of site-specific recombination events and that is flanked by two recombination site partners. The marker sequence may be a toxic gene such that only cells in which the toxic gene has been replaced by the PCR product are able to grow. Alternatively, the marker gene may produce a selectable phenotypic change in cells, such as is seen, for example, after expression of β-galactosidase. Any marker may be used that enables the selection for transfer of the PCR product into the target sequence, for example β-glucuronidase (GUS) or green fluorescent protein (GFP), or fusions between GUS and GFP. The marker gene expression may be constitutive, or it may be inducible.

According to the methods of the invention, no purification of PCR products in the PCR reaction is required before introducing them into a target sequence by site-specific recombination in vivo. Thus, aliquots from the PCR reaction may be directly transferred into a cell. Alternatively, PCR products can be purified if desired, for example, by PEG precipitation.

Typically, the PCR product is transferred into cells containing a source of one or more recombination proteins for cloning the PCR product into the target sequence. In some embodiments, a source of one or more recombination proteins is introduced into the cell along with the PCR product. The source of one or more recombination proteins is typically transient. For example, the source of recombination protein(s) may be a plasmid that is removed from the cells after the site-specific recombination reaction has occurred. Alternatively, the expression of the recombination protein(s) may be transiently induced.

The PCR product may be introduced into the cell by a high frequency method, for example by electroporation. Other methods of transferring DNA into cells are within the scope of the invention. The PCR may be introduced into a cell already comprising the target sequence, for example in the genome or on a plasmid. Alternatively, the target sequence may be introduced into the cell at the same time as the PCR product.

In some embodiments, the methods of the invention use the integrase/att system from bacteriophage lambda to direct site-specific recombination. Accordingly, the starting point for cloning PCR products is a target sequence flanked by the two recombination site partners that are attP sites, attP1 and attP2. The two attP sites flank a marker gene that permits the identification site-specific recombinants. For example, the marker gene may be ccdB, a toxin gene that is lethal in E. coli strains lacking the antitoxin, ccdA or a mutated DNA gyrase (Bahassi et al. (1999) J. Biol. Chem. 274:10936-44; Bernard et al. (1993) J. Mol. Biol. 234:534-41). If ccdB is replaced by the PCR product by site-specific recombination, the target sequence is no longer toxic to E. coli strains lacking the antitoxin. Thus, only recombinants in which the toxic gene is replaced by the PCR product will permit the growth of ccdA-minus E. coli. Alternatively, the marker gene may be any other gene that provides a phenotype that can be selected for or against. As noted above, the target sequence may be a chromosomal sequence or it may be on a plasmid.

PCR products are synthesized using PCR primers that contain the attB1 and attB2 sites. For example, the PCR products may be cloned by first adding gene specific primers, carrying out the PCR reaction for several rounds, then adding the attB primers and running the PCR reaction to completion. Nested PCR may be performed, starting with primers containing only 12 bp of the attB sequence in the first rounds of synthesis then amplifying these using primers that contain the entire attB sequence (see, e.g., http://www.invitrogen.com/). The second set of primers is standard and thus less expensive.

The PCR primers may include additional nucleotides at their ends. Lengthening the primers in this way may allow some degradation of the ends to occur without destroying the recombination sites needed for site-specific recombination. In addition, the PCR primers may contain chi sequences. Chi sequences are recognized in the functioning of certain recombination pathways in E. coli. For example, the E. coli recBCD nuclease is sequence-specific. Recognition of the chi sequences is orientation-dependent with respect to a double stranded gap that initiates recBCD binding. The presence of chi sequences, or similar sequences, on the PCR primers may enhance the efficiency of the methods of the invention (Dabert & Smith (1997) Genetics 145(4):877-89).

The PCR reaction may be carried out with a Pfx proofreading polymerase (Cline et al. (1996) Nucleic Acids Res. 24:3546-51) to keep the error rate low. To decrease the problem of incorrect priming, polymerase that is blocked by an antibody may be used (Kellogg et al. (1994) Biotechniques 16:1134-7). Thus, PCR reactions are set up at room temperature and when the temperature is raised, the antibody is denatured and the reaction begins. DNA in PCR reactions may be monitored using fluorescence. DMSO and glycerol may be used in the PCR reactions to reduce problems caused by DNA with a high G/C content (Moreau et al. (1997) Methods Mol Biol. 67:47-53; Varadaraj & Skinner (1994) Gene 140:1-5).

In some embodiments, the PCR product is transformed into E. coli. The E. coli strain typically has a mutation in the recD gene, which codes for one of the major endonucleases. It has been shown that mutating the recD gene stabilizes linear DNA. Therefore, the recD mutation may allow for better survival of the linear PCR product as it enters the cell. The E. coli strain may additionally have mutations in other genes, such as recA, ends, sbcA, sbcB, recB, or recC, in order to increase the efficiency of linear DNA uptake and survival. The presence of lambda red genes may also be desirable, as these genes enhance recombination in recombination deficient strains of E. coli (Datsenko & Wanner (2000) Proc. Natl. Acad. Sci. U.S.A. 97(12):6640-5).

The E. coli strain also contains a source of lambda integrase. Typically, the integrase gene may be provided on a plasmid under the control of an inducible promoter, for example, the lac promoter which is inducible by IPTG. The plasmid may additionally have a temperature-sensitive origin of replication such that the plasmid may be cured by incubating the cells at high temperatures. Plasmids that express lambda integrase are available (see, e.g., Platt et al. (2000) Plasmid 43:12-23).

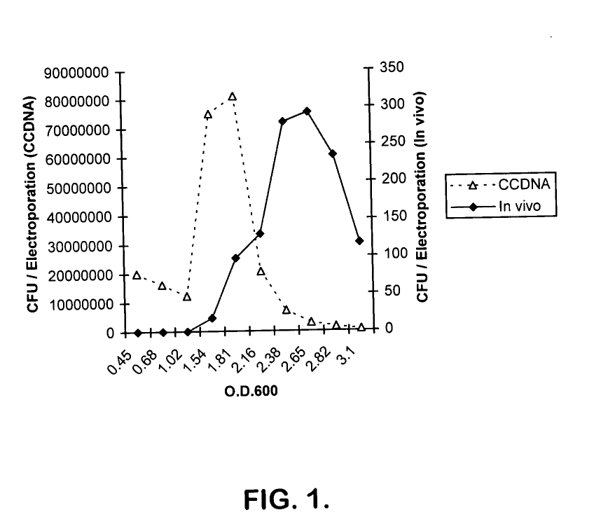

The E. coli cells expressing the lambda integrase are made competent using a modification of standard techniques, as described, for example, in EXAMPLE 1. In some embodiments, the cells are harvested during the stationary phase of growth for competence induction. As cells enter stationary phase, the expression of Integration Host Factor (IHF) is induced. IHF is required for lambda integrase function and may be responsible for the transformation frequency increase under these conditions. Therefore, the efficiency of transfer of the PCR product into the vector may be increased by using cells made competent during stationary phase, as shown in FIG. 1. Alternatively, another source of IHF or other enhancing factors may be provided. For example, the IHF coding sequences may be provided operably linked to an inducible promoter. This would permit the cells to be harvested during an earlier phase of growth, which is the peak competence stage (see FIG. 1).

The PCR product may be transformed into E. coli simultaneously with the target sequence. The target sequence may be a genomic sequence, or it may be a plasmid sequence

Alternatively, the target sequence may be transformed into E. coli first, provided that a nontoxic marker gene is used. Without being bound to any particular theory of operation, it is likely that lambda integrase recognizes the attB1 and attP1 sites, and the attB2 and attP2 sites, and mediates recombination between the respective site pairs. The recombination replaces the marker gene, for example the toxic ccdB gene, with the PCR product and creates attL1 and attL2 sites flanking the PCR product sequence.

Using the methods of the invention, the cloning of a PCR product occurs entirely in vivo, without the need for restriction enzymes, ligation enzymes, or DNA purification steps. Once the PCR product is cloned into the vector, it can then be moved into any number of other genomic sites or plasmid vectors using site-specific recombination (see, e.g., Hartley et al. (2000) Genome Res. 10:1788-95; Walhout et al. (2000) Science 287:116-22; Walhout et al. (2000) Methods Enzymol. 328:575-92). Therefore, the methods of the invention are faster, easier, less expensive, and provide a larger variety of uses than traditional PCR cloning procedures.

In some embodiments, the methods of the invention provide a frequency of cloning PCR products into a target sequence in vivo of almost 100%. Thus, almost 100% of selected cells obtained using the methods of the invention contain target sequences in which the marker gene has been replaced by the PCR product. In some embodiments, the methods of the invention provide a frequency of cloning PCR products into a target sequence in vivo of about 80%. In some embodiments, the methods of the invention provide a frequency of cloning PCR products into a target sequence between about 50% and about 95%.

In some embodiments of the invention, the methods for cloning PCR products use a site-specific recombination system other than the integrase/att system from bacteriophage lambda. Other methods may employ other bacteriophage recombination systems (e.g, φ80, P2, P4 or Cre/loxP of P1), or other non-bacteriophage recombination systems (e.g., Flp/FRT of yeast). In some embodiments, the site-specific recombination event occurs in cells other than E. coli.

In another aspect, the invention provides site-specific recombination kits for cloning PCR products by site-specific recombination in vivo. In some embodiments, the kits of the invention comprise competent cells of a genotype able to carry out the recombination reaction (e.g., V355(pBH-INT)), a suitable vector plasmid (e.g., pMK2010), a control PCR product or primers sufficient for generating such PCR product. The kits may also include a set of secondary primers (attB primers) for a nested PCR protocol. These primers may contain modified DNA bases or intrabase linkages.

### EXAMPLE 1

This Example describes a representative method of the invention using the integrase/att system from bacteriophage lambda as the site-specific recombination system.

PCR products were generated for the lacZa gene of E. coli using primers that cause the product to be flanked by bacteriophage lambda attB1 and attB2 sequences. Two sets of primers were used (primary and secondary) in a nested PCR protocol. The primary primer sequences were 5′ GGA GGC TCT TCA ATG ACC ATG ATT ACG GAT TC 3′ (SEQ ID NO:1) and 5′ AGC TGG GTT CTA CGC CGA GTT AAC GCC ATC AA 3′ (SEQ ID NO:2). The secondary sequences were 5′ GGG GAC AAG TTT GTA CAA AAA AGC AGG CTT AGG AGG CTC TTC AAT G 3′ (SEQ ID NO:3) and 5′GGG GAC CAC TTT GTA CAA GAA AGC TGG GTU CTA 3′ (SEQ ID NO:4). The PCR product and the plasmid vector pMK2010 were co-electroporated into electrocompetent cells of E. coli strain V355 (pBH-INT). V355 (CGSC6720) is a recD mutant, inactivating one of the major exonucleases of E. coli. Plasmid vector pMK2010 contains the ccdB gene, which is toxic to most E. coli strains, flanked by attP1 and attP2 sites. pBH-INT contains the gene encoding lambda integrase under control of the lactose operon repressor (i.e., inducible by IPTG), and a temperature-sensitive origin of replication.

V355 (pBH-INT) electrocompetent cells were prepared by growing an overnight culture in LB Broth containing 100 μg/ml ampicillin, diluting the culture 1:100 into LB Broth containing 100 μg/ml ampicillin and 20 μg/ml IPTG, and growing the culture by shaking at 30° C. and 240 r.p.m. until the OD₆₀₀ reached 2.4 to 2.6 (about 4 to 6 hours). The cells were prepared for electroporation by successive washes in 10% glycerol, and the final cell pellet were resuspended in 1/250 of the original culture volume of 10% glycerol. At this point, the cells were distributed into 45 to 90 μl aliquots, frozen with liquid nitrogen, and stored at −80° C.

3 μl of the PCR product (3000 ng/microliter) and 1 μL of pMK2010 (150 ng/microliter) were co-electroporated into a 50 μl aliquot of competent cells. The cells were grown out in SOC medium at room temperature, which is the optimal temperature for lambda integrase function, then plated on onto LB Broth plates containing 75 μg/ml kanamycin to select for the pMK2010 derivative, and incubated at 42° C. overnight to cure the cells of the pBH-INT plasmid. Between 400-600 recombinant clones were obtained per electroporation. Based on restriction enzyme analyses, 23 out of 24 clones contained correctly cloned PCR products.

Cells grown to different culture densities were prepared for electroporation and transformed either with control plasmid to test cell competence for transformation with supercoiled DNA or transformation with vector plasmid and the PCR product containing the attB sequences. Even though peak electrocompetence occurs when cells are harvested during late logarithmic growth phase, peak numbers of cloned PCR products were obtained when cells were well into stationary phase, at an OD₆₀₀ of about 2.5, as shown in FIG. 1. The peak represents ˜300 cloned PCR products per electroporation. The data show that successful transformation leading to in vivo recombination requires a different physiological state of the cell than does transformation with supercoiled DNA, with maximum competence for recombination occurring much later in cell growth. This may be due to the presence of integration host factor (IHF), which is required for integrase function, in stationary phase cells. Consistent with this, expression of IHF in early growth phase produces a better efficiency of transformation with cloned PCR products in early growth phase and peak numbers of cloned PCR products are obtained earlier in the growth phase.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for cloning a polymerase chain reaction (PCR) product into a target sequence, comprising transferring a PCR product into a target sequence using a site-specific recombination system in vivo.

2. The method of claim 1, wherein the transfer of the PCR product into the target sequence comprises the steps of:
providing a PCR product flanked by a first site-specific recombination site and a second site-specific recombination site; and
transferring the PCR product into a cell comprising a target sequence flanked by a first recombination site partner and a second recombination site partner and at least one recombination protein that mediates recombination between the first site-specific recombination site and the first recombination site partner and between the second site-specific recombination site and the second recombination site partner.

3. The method of claim 1, wherein the target sequence is a plasmid sequence.

4. The method of claim 1, wherein the target sequence is a genomic sequence.

5. The method of claim 2, wherein the PCR product is generated using a set of primers to provide the first and second recombination sites to the PCR product.

6. The method of claim 2, wherein the target sequence is transferred into the cell simultaneously with the PCR product.

7. The method of claim 1, wherein the site-specific recombination system is the integrase/att system from bacteriophage lambda.

8. The method of claim 2, wherein the cell is a bacterial cell.

9. The method of claim 7, wherein the bacterial cell is an E. coli cell.

10. The method of claim 2, wherein at least one of the recombination proteins comprises lambda integrase.

11. The method of claim 10, wherein at least one of the recombination proteins comprises Integration Host Factor.

12. The method of claim 11, wherein Integration Host Factor is present in the cell in early growth phase.

13. The method of claim 4, wherein the first site-specific recombination site is attB1 site, wherein the second site-specific recombination site is an attB2 site, wherein the first recombination site partner is an attP1 site, and wherein the second recombination site partner is an attP2 site.

14. The method of claim 1, wherein the PCR product is linear.

15. The method of claim 1, wherein the PCR product is cloned without purification from the PCR reaction.
